(19) United States
(12) Patent Application Publication
(10) Pub. No.: US 2006/0079500 A1
(43) Pub. Date: Apr. 13, 2006

(54) Medicament and method for reducing alcohol and/or tobacco consumption

(75) Inventors: Joachim Moormann, Werne (DE); Klaus Opitz, Munster (DE); Hermann Mucke, Wien (AT)

Correspondence Address: BIRCH STEWART KOLASCH & BIRCH, PO BOX 747, FALLS CHURCH, VA, 22040-0747, US

(73) Assignee: HF PHARMACEUTICAL INC., WERNE (DE)

(21) Appl. No.: 10/523,012

(22) PCT Filed: Jul. 25, 2003
(86) PCT No.: PCT/EP03/08236

§ 371 Date: Feb. 1, 2005

(30) Foreign Application Priority Data
Aug. 3, 2002 (DE) 102 35 556.8

(51) Int. Cl. A61K 31/55; A61K 31/4439

(52) U.S. Cl. 514/214.03; 514/343

(57) ABSTRACT

The present invention relates to a medicament for treating addiction craving which is characterized in that the medicament consists of a combination of two administration forms, one of the administration forms continuously releasing at least one modulator of nicotinic receptors and the other administration form enabling a rapid entry of galanthamine into the central nervous system, as well as a two-stage method for the therapy of substance craving through modulation of neuronal nicotinic receptors, wherein a permanent treatment with a pharmaceutical administration form which continuously delivers a modulator of nicotinic receptors is supplemented upon the appearance of a strong craving for a substance by administering galanthamine or a pharmacologically acceptable salt thereof by means of an administration form which enables rapid entry of this modulator into the central nervous system.

The subject-matter of the present invention is to provide a medicament for the therapy of addiction craving which enables separate or simultaneous reduction of concurrent alcohol and nicotine abuse, with particular consideration being given to relapse-promoting breakthrough craving.

The present invention in particular relates to a medicament for the therapy of addiction craving consisting of two different administration forms to enable an improved therapy of addiction craving in the case of alcohol and/or nicotine abuse, with particular consideration being given to relapse-promoting breakthrough craving.

It is also a subject matter of the present invention to provide a medicament for the therapy of addiction craving which enables separate or simultaneous reduction of concurrent alcohol and nicotine abuse with particular consideration being given to relapse-promoting breakthrough craving, and the application of which does not seriously impair the patient's activities at work or his social activities.

A further subject matt of the present invention relates to a therapeutic metho or separate or simultaneous reduction of concurrent alcohol and nicotine abuse, wherein particular consideration is given to relapse-promoting break-through craving.

Furthermore, it is a subject matter of the invention to render this therapeutic method as a two-phase method, so that it is possible, on the one hand, to counteract the basic craving for alcohol and/or tobacco products and, on the other hand, to counteract the breakthrough craving which occurs against this background, with different approaches of therapy.

Recently, pharmacology has increasingly concerned itself with the symptom complex of substance craving, and has offered an explanation in the molecular range for numerous behavioural phenomena connected with substance craving which could heretofore not be satisfactorily explained. Neurotransmitters, their binding to neuronal receptors, and the modulation of conduction brought about as a consequence of this interaction, are meanwhile considered the basis for an explanation of addiction behaviour.

For a relatively long time the pharmacological model concepts of addiction behaviour attached importance almost exclusively to the dopaminergic system. The model of “dopamine deficiency syndrome” was even postulated as the hypothetic generalised basis for addiction-predominated behaviour in the wider sense, i.e. also of forms of behaviour not connected with substance consumption—by analogy to the “serotonine deficiency syndrome”, which is used to explain depression and compulsive acts. Only secondary importance was attached to adrenergic and serotonergic conduction systems, especially in connection with depression and addiction behaviour.

In respect of the craving for alcohol and tobacco products, the view which subordinates the significance of the adrenergic and serotonergic conduction systems has in recent years been considerably expanded, however. A substantial part of this more thorough understanding is due to the discussion of the role of presynaptic nicotinic receptors, whose natural ligand is the neurotransmitter acetylcholine.

In the cholinergic systems the presynaptic acetylcholinergic receptors serve as autoreceptors, that is, they block the further release of acetylcholine as soon as its concentration in the synapse reaches a limit value. Nicotinic autoreceptors are, however, found in the dopaminergic, adrenergic and serotonergic conduction systems where they, in a comparable manner, modulate the release of the respective neurotransmitters as soon as acetylcholine, nicotine or another ligand binds to them.

Neuronal nicotinic receptors thus couple the cholinergic conduction system to the conduction systems which use dopamine, norepinephrine or serotonine as messenger substances. It therefore appears plausible to assume that an increase in the cholinergic tone—as can be accomplished by the administration of medicaments which increase the concentration of acetylcholine either by raising its release or by inhibiting the degrading enzyme acetylcholinesterase—should also, indirectly via nicotinic receptors, influence emotional states and the craving for alcohol and/or nicotine.

The role of neuronal nicotinic receptors as synapses between conduction systems working with different neurotransmitters also enables a re-evaluation of the known fact that alcoholics as well as so-called “high-risk drinkers” who although not fulfilling all criteria of alcohol addiction according to the currently valid schemata of the “International Classification of Disease” (ICD-10) do nevertheless constantly exceed the limit values established for alcoholism by the World Health Organisation, and are almost always heavy smokers at the same time. According to the present state of the art, alcohol and/or nicotine abuse are to a considerable extent consequences of a joint fundamental dysregulation of neuronal nicotinic receptors.

Despite the joint effects on the neuronal nicotinic receptors due to alcohol and/or nicotine abuse, a direct and sustained mutual substitution of alcohol and nicotine has neither been proven in the animal model nor in human addiction behaviour. The pharmacological administration of nicotine, i.e. through transdermal application, as a chewing gum or by means of an inhalator, for example, generally has a negligible influence on alcohol abuse. On the other hand it is generally known that withdrawal of alcohol can have both a positive and a negative influence on simultaneously existing smoking behaviour.

An explanation for this phenomenon is that nicotinic receptors do not function as simple “on-off switches”, but instead are subject to the so-called allosteric modulation due to their molecular structure. Allosteric modulation means that the response to the binding of a ligand depends on the momentary configuration of the receptor, which is in turn influenced by ligands binding at other sites.

In addition, in both forms of addiction the currently usual pharmacological relapse prevention, as practiced in smoking withdrawal by nicotine patch or nicotine chewing gum, or by acamprosat or naltrexon in alcohol withdrawal, is extremely poor. Relapses into alcohol or nicotine consumption or into concurrent abuse are normal in the first weeks and months following the withdrawal. Typically the relapses occur within the framework of a so-called “breakthrough behaviour”, which means the yielding to a suddenly occurring massive craving for a substance against the background of an otherwise controllable craving. This breakthrough craving is triggered by stress, social stimuli, the sight or smell of alcoholic beverages or cigarette smoke or the like.

It therefore seems plausible, at first sight, to combat acute craving, which occurs within the framework of a pharmacologically assisted abstinence, by quickly administering an additional dose of the relapse-delaying agent concerned, for example by using a nicotine-containing chewing gum in addition to the transdermally administered nicotine base dose according to needs. However, it is known to those skilled in the art that this route does by no means promise success but rather is potentially dependence-increasing and possibly even dangerous. Thus, nicotine which has been rapidly introduced in the central nervous system to a large extent desensitizes the nicotinic receptors located there within seconds up to minutes. For this reason, smoking, through which nicotine enters the brain more rapidly than would be possible via the intravenous route, is habit-forming, whereas transdermally administered nicotine leads to a by far slower increase in nicotine concentration in the brain and does not have a comparably high dependence potential.

This at first sight plausible therapeutic approach would therefore only replace the dependence on tobacco products by a dependence on a nicotine-containing withdrawal agent. Analogous observations can be made also for the therapy of alcohol craving.

Although a combined therapy of alcohol and/or nicotine abuse by way of influencing the nicotinic receptors would be extremely desirable from a health point of view, down to this day no such therapy exists. In particular, the “break-through craving” for alcohol and/or nicotine has not received even remotely adequate pharmacological treatment.

It has now been found, surprisingly and in light of the above by no means predictable for those skilled in the art that a two-component therapy of the alcohol and/or nicotine craving is nevertheless possible, provided that at least the treatment of the acutely appearing breakthrough craving for alcoholic beverages and/or tobacco products is carried through with the active substance galanthamine or one of its pharmacologically acceptable salts.

It was therefore an object of the present invention to provide a medicament for the therapy of alcohol and/or tobacco addiction, especially for carrying through the two-component therapy for treating the craving for alcohol and/or nicotine.

The medicament according to the invention consists of a combination of two administration forms of which one continuously delivers at least one modulator of nicotinic receptors to the patient, and the other administration form enables a rapid entry of galanthamine or of one of its pharmacologically acceptable salts into the central nervous system.

The administration form continuously delivering the modulator or modulators of nicotinic receptors serves for the therapy of the basic craving for alcohol and/or nicotine. With the administration form enabling a rapid entry of galanthamine or its pharmacologically acceptable salts it is possible to treat the craving for alcohol and/or nicotine which occurs suddenly in spite of the basic therapy.

The modulator of nicotinic receptors in the administration form which continuously delivers this modulator or these modulators is preferably selected from the group consisting of galanthamine, the pharmacologically acceptable salts of galanthamine, nicotine and the pharmacologically acceptable salts of nicotine. For the administration form continuously delivering the modulator, galanthamine is especially preferred.

The nicotinic receptors continuously releasing the modulator or modulators may be a subcutaneous implant or an intramuscularly injectable preparation which have a long-lasting depot effect. Suitable as intramuscularly injectable preparations having a long-lasting depot effect are, for example, suspensions of microcapsules containing the modulator(s) of nicotinic receptors. Especially preferred administration forms for continuous delivery are transdermal therapeutic systems.

The delivery rate of the administration form continuously releasing the modulator or the modulators of nicotinic receptors is in the range of between 10 mg and 25 mg of galanthamine or of one of the pharmacologically acceptable salts of galanthamine, or between 5 mg and 50 mg of nicotine or of one of the pharmacologically acceptable salts of nicotine, per day.

The administration forms enabling a rapid entry of galanthamine or its pharmacologically acceptable salts into the central nervous system contain between 1 mg and 5 mg of galanthamine or at least a pharmacologically acceptable salt of galanthamine.

Suitable as administration forms enabling a rapid entry of galanthamine or its pharmacologically acceptable salts into the central nervous system are solid biocompatible matrices which are rapidly disintegratable in saliva, for example having the shape of a post stamp, or solutions which are dropped or sprayed into the nasal cavity or are kept in the oral cavity.

The solutions for pernasal or buccal administration may be in the form of a flexible plastic vessel, with a capacity of between 1 and 5 ml, the said plastic vessels for the formulations to be sprayed or dropped into the nose being provided with appropriately formed nozzles.

According to the invention, the combination therapy for treating addiction behaviour is preferably adapted such that the therapy of the basic craving for alcohol and/or nicotine is carried through by creating a galanthamine concentration in the central nervous system which is as uniform as possible, by means of an administration form which delivers galanthamine slowly and continuously. According to the invention, the craving for alcohol and/or nicotine, which in such a basic therapy nevertheless suddenly occurs, is treated exclusively with galanthamine or one of its pharmacologically acceptable salts, in such a manner that a rapid absorption of galanthamine or its pharmacologically acceptable salt through the mucosa of the oral cavity or nose is possible.

Galanthamine (4a,5,9,11,12-hexahydro-3-methoxy-11-methyl[6H]-benzofuro-[3a,3,2ef][2]benzazepin-6-ol) is approved in the form of tablets and a drinking solutions under the tradename Reminylo for the treatment of Alzheimer's dementia. Galanthamine modulates neuronal nicotinic receptors through two different mechanisms: indirectly by increasing the synaptic concentration of acetylcholine by means of inhibiting the acetylcholinesterase, and directly as a so-called allosterically potentiating ligand (APL).

The use of galanthamine or its pharmacologically acceptable acid addition salts as a means to fight substance craving is in principle known. Its use in the therapy of alcohol abuse is disclosed in the patent specification DE 4010079.

Also, galanthamine-containing transdermal therapeutic systems are known from the patent DE 4301783. Such a transdermal therapeutic system, employed as a sole therapy of relapsing alcoholics, in a clinical study decreased the alcohol consumption in a relapse, but it accelerated the onset of the relapse compared to the placebo. It follows therefrom that the exclusive use of transdermally administered galanthamine in such patients does not represent an optimum therapy solution.

The use of galanthamine or its pharmacologically acceptable acid addition salts for combating nicotine consumption is claimed in the patent DE 4301782. The above-mentioned clinical study provided indications in the case of those test subjects who also were smokers that there was a relatively small decrease in cigarette consumption, which occurred, however, only after several weeks of therapy exclusively with transdermally administered galanthamine. Again, the sole use of transdermally administered galanthamine does not seem to be optimal for achieving the therapeutic aim.

None of the above-referenced documents mentions the use of galanthamine in a form which rapidly enters the brain for preventing or treating breakthrough craving against the background of a long-term therapy with galanthamine or another substance modifying the craving for alcohol. On the contrary, in those documents galanthamine is discussed and claimed exclusively with emphasis on its suitability for long-term therapy, especially utilizing administration forms causing a retarded release.

In the therapeutic praxis the method of treating substance craving according to the invention is applied such that the person to be treated, who can be either clinically alcohol dependent with ongoing consumption of alcohol, an alcoholic under withdrawal therapy or after its conclusion, or who may be abusing alcohol in the sense of the WHO criteria, with the persons of any of these groups possibly also consuming tobacco products, is given a basic therapy with a nicotinic agonist, independently of whether the person concerned was consuming tobacco products at the start of the therapy. The base therapy is preferably carried out by means of a galanthamine-containing transdermal therapeutic system releasing between 10 mg and 25 mg of galanthamine per day, but it may also be realized by subcutaneous implants or intramuscularly injected preparations with long-lasting depot effect which have an appropriate delivery rate. Injectable suspensions of microencapsulated active substance are known to those skilled in the art, especially for use with psychopharmaceuticals, for example by means of the microparticular formulations according to U.S. Pat. No. 6,264.987 or WO 00/35423. Likewise, biocompatible, degradable subcutaneous implants are known, for example, from U.S. Pat. No. 6,312,708.

As an alternative, the basic therapy may also be carried out with commercial nicotine-containing transdermal therapeutic systems. Transdermal therapeutic systems containing nicotine as the sole active substance for combating tobacco consumptions are described, for example, in the German patent DE 3629304 or in U.S. Pat. No. 4,597,961. Suitable in the sense of the present invention are also the transdermal systems claimed in WO 01/80837 which contain combinations of nicotine or its salts, optionally with additional use of nicotinically active substances such as lobeline or succinylcholine, and compounds having antidepressive action.

If the person thus treated notices a suddenly appearing craving for alcohol and/or tobacco products, or is on the point of exposing himself to a situation where according to experience the occurrence of a craving for alcohol and/or tobacco products is to be expected, for example when participating in events where people drink alcohol and smoke, the person administers to himself 1 to 5 mg of galanthamine in a form which, by transmucosal absorption, effects a safeguard against the breakthrough craving which starts within minutes and is active for 2 to 3 hours. It is essential that this rapidly acting medication is easy to carry along and that its administration can be carried out in a quick, painless, socially inconspicuous manner and without recourse to auxiliaries.

According to the invention, the administration can be carried out by uptake of a buccal solution from a flexible polymer container with a capacity of 1 to 5 ml into the oral cavity, or by uptake of a galanthamine-containing, biocompatible solid matrix rapidly soluble in saliva, for example having the shape of a post stamp, or by spraying or dropping appropriately formulated solutions into the nose using flexible plastic containers with appropriately formed nozzles, all of the above being equivalent. Suitable solid buccal administration forms are, for example, those described in DE 199 13 731 or DE 196 52 188. Pernasal formulations, which are suitable, in particular, for alkaloids, are also widely known among those skilled in the art, thus for morphine, for example, from WO 00/76506.

1. Medicament for treating addiction craving, characterized in that the medicament consists of a combination of two administration forms, one of the administration forms continuously releasing at least one modulator of nicotinic receptors and the other administration form, which can be administered independently from the first-mentioned administration form, enabling a rapid entry of galanthamine or one of its pharmacologically acceptable salts into the central nervous system.

2. Medicament according to claim 1, characterized in that the modulator of nicotinic receptors in the administration form continuously releasing the modulator is selected from the group consisting of galanthamine, the pharmacologically acceptable salts of galanthamine, nicotine and the pharmacologically acceptable salts of nicotine, with galanthamine being preferred.

3. Medicament according to claim 1, characterized in that the administration form continuously releasing the modulator or the modulators of nicotinic receptors is selected from the group consisting of transdermal therapeutic systems, subcutaneous implants and intramuscularly injectable preparations.

4. Medicament according to claim 3, characterized in that the intramuscularly injectable preparation is a suspension of microcapsules containing the modulator or the modulators of nicotinic receptors.

5. Medicament according to claim 3, characterized in that the administration form continuously releasing the modulator or modulators of nicotinic receptors releases between 10 mg and 25 mg of galanthamine or a pharmacologically acceptable salt of galanthamine, or between 5 mg and 50 mg of nicotine or a pharmacologically acceptable salt of nicotine, per day.

6. Medicament according to claim 1, characterized in that the administration form enabling a quick entry of galanthamine or a pharmacologically acceptable salt of galanthamine into the central nervous system contains galanthamine or a pharmacologically acceptable salt of galanthamine in an amount of from 1 to 5 mg.

7. Medicament according to claim 1, characterized in that the administration form enabling a quick entry of galanthamine or a pharmacologically acceptable salt of galanthamine into the central nervous system is selected from the group consisting of solid, biocompatible matrices quickly soluble in saliva, buccal solutions, as well as spray or drip solutions.

8. Medicament according to claim 7, characterized in that the administration form for solutions which enables a rapid entry of galanthamine or a pharmacologically acceptable salt of galanthamine into the central nervous system is a flexible plastic container with a capacity of between 1 and 5 ml.

9. Medicament according to claim 8, characterized in that the plastic container is provided with nozzles through which the solution can be sprayed or dripped into the nose.

10. Method for treating substance craving by modulation of neuronal nicotinic receptors, characterized in that it is a two-stage method wherein a permanent treatment with a pharmaceutical administration form which continuously delivers a modulator of nicotinic receptors is supplemented upon the appearance of a strong craving for a substance by administering galanthamine or a pharmacologically acceptable salt thereof by means of an administration form which enables rapid entry of galanthamine or of a pharmaceutically acceptable salt thereof into the central nervous system.

11. Method according to claim 10, characterized in that the substance craving is a craving for alcoholic beverages and/or tobacco products.

12. Method according to claim 10, characterized in that the modulator of nicotinic receptors in the administration form releasing the modulator continuously is selected from the group consisting of galanthamine, the pharmacologically acceptable salts of galanthamine, nicotine and the pharmacologically acceptable salts of nicotine, with galanthamine being preferred.

13. Method according to claim 10, characterized in that the administration form releasing the modulator or the modulators of nicotinic receptors continuously is selected from the group consisting of transdermal therapeutic systems, subcutaneous implants and intramuscularly injectable preparations.

14. Method according to claim 13, characterized in that the subcutaneously injectable preparation is a suspension of microcapsules containing the modulator or modulators of nicotinic receptors for intramuscular injection.

15. Method according to claim 13, characterized in that the administration form continuously releasing the modulator or modulators of nicotinic receptors releases between 10 mg and 25 mg of galanthamine or a pharmacologically acceptable salt of galanthamine, or between 5 mg and 50 mg of nicotine or a pharmacologically acceptable salt of nicotine, per day.

16. Method according to claim 10, characterized in that the administration form enabling a quick entry of galanthamine or of a pharmacologically acceptable salt of galanthamine into the central nervous system contains galanthamine or a pharmacologically acceptable salt of galanthamine in an amount of from 1 to 5 mg.

17. Method according to claim 10, characterized in that the administration form enabling a rapid entry of galanthamine or of a pharmacologically acceptable salt of galanthamine into the central nervous system is selected from the group consisting of solid, biocompatible matrices rapidly soluble in saliva, biocompatible matrices, buccal solutions, as well as spray and drip solutions.

18. Method according to claim 17 characterized in that the administration form for solutions which enables a rapid entry of galanthamine or of a pharmacologically acceptable salt of galanthamine into the central nervous system is a flexible plastic container with a capacity of between 1 and 5 ml.

19. Method according to claim 18 characterized in that the plastic container is provided with nozzles through which the solution can be sprayed or dripped into the nose.
